(19) United States
(12) Patent Application Publication
(10) Pub. No.: US 2016/0106511 A1
(43) Pub. Date: Apr. 21, 2016

(54) SURGICAL STERILIZATION DEVICE AND METHODS OF USE

(71) Applicant: Barry University, Miami Shores, FL (US)

(72) Inventors: Jeffrey L. Jensen, Palm City, FL (US); Mario Cala, Miami, FL (US); Romain Onteniente, North Miami, FL (US)

(21) Appl. No.: 14/884,258

(22) Filed: Oct. 15, 2015

Related U.S. Application Data
(60) Provisional application No. 62/064,925, filed on Oct. 16, 2014.

(51) Int. Cl. A61B 19/00; A61F 13/40; A61L 2/00

(52) U.S. Cl. CPC A61B 19/36; A61L 2/0088; A61M 35/006; A61B 2019/086

(57) ABSTRACT

Embodiments of the present disclosure include materials and methods relating to sterilization devices. In certain embodiments, materials and methods disclosed herein relate to pre-surgical sterilization of an appendage of a subject using a device that reduces surgical preparation time and microbial growth. Certain embodiments of the present disclosure disclose materials and methods to ameliorate high costs associated with medical care, especially the utilization of costly elective hospital services, by for example reducing operating room costs and increasing efficiency without compromising patient health.

### RELATED APPLICATIONS

The instant application claims the benefit of U.S. Provisional Patent Application Ser. No. 62/064,925, filed Oct. 16, 2014. This application is incorporated herein by reference in its entirety for all purposes.

### FIELD

Embodiments of the present disclosure include materials and methods relating to sterilization devices. More specifically, the present disclosure relates to the pre-surgical sterilization of an appendage of a subject using a device that reduces surgical preparation time and microbial growth.

### BACKGROUND

Hospitals and health care systems throughout the world are continually asked to increase the quality of patient care, while also reducing costs. Achieving this goal is particularly challenging in the operating room, where both the efficiency and the cost of care for routine, elective, and emergency situations constitute a significant and growing medical problem. Surgical procedures typically involve high labor costs, which are exacerbated by nursing shortages, and this in turn makes it more difficult to increase operating room efficiency without compromising patient health or increasing the potential for infection.

Equally problematic is the unavailability of devices to assist in allowing surgical procedures to be moved from the main operating rooms to a minor procedure room or to an office setting. Often times the logistics in providing a sterile, efficient environment where a patient's welfare is not compromised is the main deterrent for moving surgical procedures from the main operating rooms to other less expensive settings. Consequently, operating room costs for minor procedures can be fivefold that of surgeon's costs, and these costs are often passed on to the patient.

### SUMMARY

Aspects of the present disclosure include a sterilization device for sterilizing or disinfecting an area of a subject's body. In some aspects, the sterilization device can include a multilayered transparent sleeve having an outer flexible layer and an inner impermeable layer; an absorbent foam insert that includes a plurality of sterilization sacs containing one or more sterilization agents, the absorbent foam insert positioned within the multilayered transparent sleeve and configured for placement against an area of a subject's body in need of medical treatment; and one or more stabilization mechanisms for securing the sterilization device to the subject's body. In some aspects, at least one of the plurality of sterilization sacs is configured to release the one or more sterilization agents upon application of pressure to the sterilization sac onto a targeted region of a subject's body in need of medical treatment.

A device according to paragraph [0005], wherein the inner impermeable layer includes a substantially transparent polymeric material.

A device according to either paragraph [0005] or [0006], wherein the inner impermeable layer includes a polyurethane material.

A device according any of paragraphs [0005]-[0007], further including a breaker configured to engage at least one of the plurality of sterilization sacs upon application of pressure to the breaker to facilitate release of the one or more sterilization agents from the at least one of the plurality of sterilization sacs.

A device according any of paragraphs [0005]-[0008], further including a breaker having one or more structures or components that includes a pointed structure, a perforation, and a valve.

A device according any of paragraphs [0005]-[0009], wherein the absorbent foam insert includes open cell foam.

A device according any of paragraphs [0005]-[0010], wherein the one or more sterilization agents include one or more of hexidine, chlorhexidine, betadine, iodine, alcohol and derivatives and combinations thereof.

A device according any of paragraphs [0005]-[0011], wherein the one or more stabilization mechanisms include one or more of adhesive strips, drawstrings, and metal strips.

A device according any of paragraphs [0005]-[0012], wherein the multilayered transparent sleeve and the absorbent foam insert are coupled so that the breaker is positioned at least partially over the sterilization sac.

A device according any of paragraphs [0005]-[0013], further including a tourniquet having a bladder and a valve through which the tourniquet can be inflated.

Aspects of the present disclosure can include methods of sterilizing an area of a subject's appendage. In some aspects, the methods can include positioning a sterilization device over an area of the subject's appendage or body part in need of medical treatment, the sterilization device comprising: a transparent sleeve having an outer layer and an inner impermeable layer; an absorbent foam insert comprising a plurality of sterilization sacs containing one or more sterilization agents; and one or more stabilization mechanisms. In some aspects, the method can include securing the sterilization device to the area of the subject's appendage or body part in need of medical treatment using the one or more stabilization mechanisms. In some aspects, methods disclosed herein can include applying pressure to one or more of the plurality of sterilization sacs to release the one or more sterilization agents into the absorbent foam insert. In other aspects, methods can include applying mechanical force to the outer layer of the sterilization device to distribute the one or more sterilization agents through the foam insert to the area of the subject's appendage or body part in need of medical treatment.

A method according to paragraph [0015], wherein the inner impermeable layer includes a polyurethane material or similar material flexible plastic material. In other embodiments, gauze or gauze-like material can be used.

A method according to either paragraph [0015] or [0016], wherein pressure can be applied to one or more breaker structures or mechanisms having at least one of pointed structures, perforations, or valves in order to release the one or more sterilization agents from the plurality of sterilization sacs.

A method according to any of paragraphs [0015]-[0017], wherein the absorbent foam insert includes open cell foam. In accordance with these embodiments, open cell foam can include open cell polyurethane foam or similar foam.

A method according to any of paragraphs [0015]-[0018], wherein the one or more sterilization agents includes one or more of hexidine, chlorhexidine, betadine, iodine, and derivatives and combinations thereof.

A method according to any of paragraphs [0015]-[0019], wherein the sleeve and the foam insert are coupled so that a plurality of breakers in an outer sleeve layer align with the plurality of sterilization sacs in the foam insert.

A method according to any of paragraphs [0015]-[0020], wherein positioning the sterilization device further includes aligning a plurality of breakers in an outer layer of the sleeve with the plurality of sterilization sacs in the foam insert.

A method according to any of paragraphs [0015]-[0021], wherein the method can further include inflating a bladder of a tourniquet wherein the tourniquet is coupled to the sterilization device.

Certain aspects of the present disclosure can include a surgical sterilization kit. In some aspects, the surgical sterilization kit can include a multilayered transparent sleeve having an outer flexible layer and an inner impermeable layer; an absorbent foam insert that includes a plurality of sterilization sacs containing one or more sterilization agents; and one or more stabilization mechanisms for securing the sterilization device to an area of a subject's appendage in need of medical treatment. In some aspects, the application of pressure to the sterilization sacs releases the one or more sterilization agents from the plurality of sterilization sacs and sterilizes the area of the subject's appendage in need of medical treatment.

A sterilization kit according to paragraph [0023], further including a plurality of breaker structures or mechanisms having one or more of pointed structures, perforations, or valves that facilitate release of the one or more sterilization agents from the plurality of sterilization sacs.

A sterilization kit according to either paragraph [0023] or [0024], further including a tourniquet and a source of compressed air or carbon dioxide for inflating the tourniquet.

These and other advantages will be apparent from the disclosure of the aspects, embodiments, and configurations contained herein.

### DEFINITIONS

As used herein, “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together. When each one of A, B, and C in the above expressions refers to an element, such as X, Y, and Z, or class of elements, such as X1-Xn, Y1-Ym, and Z1-Zo, the phrase is intended to refer to a single element selected from X, Y, and Z, a combination of elements selected from the same class (for example, X1 and X2) as well as a combination of elements selected from two or more classes (for example, Y1 and Zo).

It is to be noted that the term “a” or “an” entity refers to one or more of that entity. As such, the terms “a” (or “an”), “one or more” and “at least one” can be used interchangeably herein. It is also to be noted that the terms “comprising”, “including”, and “having” can be used interchangeably.

The term “means” as used herein shall be given its broadest possible interpretation in accordance with 35 U.S. C. Section 112(f). Accordingly, a claim incorporating the term “means” shall cover all structures, materials, or acts set forth herein, and all of the equivalents thereof. Further, the structures, materials or acts and the equivalents thereof shall include all those described in the summary, brief description of the drawings, detailed description, abstract, and claims themselves.

It should be understood that every maximum numerical limitation given throughout the present disclosure is deemed to include each and every lower numerical limitation as an alternative, as if such lower numerical limitations were expressly written herein. Every minimum numerical limitation given throughout the present disclosure is deemed to include each and every higher numerical limitation as an alternative, as if such higher numerical limitations were expressly written herein. Every numerical range given throughout the present disclosure is deemed to include each and every narrower numerical range that falls within such broader numerical range, as if such narrower numerical ranges were all expressly written herein.

The preceding is a simplified summary of the disclosure to provide an understanding of some aspects of the disclosure. This summary is neither an extensive nor exhaustive overview of the disclosure and its various aspects, embodiments, and configurations. It is intended neither to identify key or critical elements of the disclosure nor to delineate the scope of the disclosure but to present selected concepts of the disclosure in a simplified form as an introduction to the more detailed description presented below. As will be appreciated, other aspects, embodiments, and configurations of the disclosure are possible utilizing, alone or in combination, one or more of the features set forth above or described in detail below.

### BRIEF DESCRIPTION OF THE DRAWINGS

The accompanying drawings are incorporated into and form a part of the specification to illustrate several examples of the present disclosure. These drawings, together with the description, explain the principles of the disclosure. The drawings simply illustrate preferred and alternative examples of how the disclosure can be made and used and are not to be construed as limiting the disclosure to only the illustrated and described examples. Further features and advantages will become apparent from the following, more detailed, description of the various aspects, embodiments, and configurations of the disclosure, as illustrated by the drawings referenced below.

FIG. 1A is a representative illustration of a sterilization device, according to an embodiment of the present disclosure.

FIG. 1B is a representative illustration of a longitudinal cross-sectional view of a sterilization device, according to an embodiment of the present disclosure.

FIG. 1C is a representative illustration of a cross-sectional view of a sterilization device in a rolled up configuration, according to an embodiment of the present disclosure.

FIG. 1D is a representative illustration of a sterilization device coupled to a tourniquet, according to an embodiment of the present disclosure.

FIG. 2A is a representative illustration of a sterilization device having two separate stabilization mechanisms, according to an embodiment of the present disclosure.

FIG. 2B is a representative illustration of a sterilization device having horizontally aligned breakers and sterilization sacs, according to an embodiment of the present disclosure.

FIG. 2C is a representative illustration of a sterilization device having no centrally located breakers and sterilization sacs, according to an embodiment of the present disclosure.

FIG. 2D is a representative illustration of a sterilization device having peripherally located breakers and sterilization sacs, according to an embodiment of the present disclosure.

FIG. 3 is a representative illustration of several different configurations of the sterilization device on a subject's body, according to an embodiment of the present disclosure.

FIG. 4 is a flowchart representing a method of sterilizing a portion of a subject's appendage, according to an embodiment of the present disclosure.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure.

### DETAILED DESCRIPTION

Embodiments of the present disclosure include materials and methods relating to sterilization devices. In certain embodiments, materials and methods disclosed herein relate to pre-surgical sterilization of an appendage or body part of a subject using a device that reduces surgical preparation time and microbial growth. In accordance with these embodiments, methods and uses of these sterilization devices can be performed by a healthcare professional.

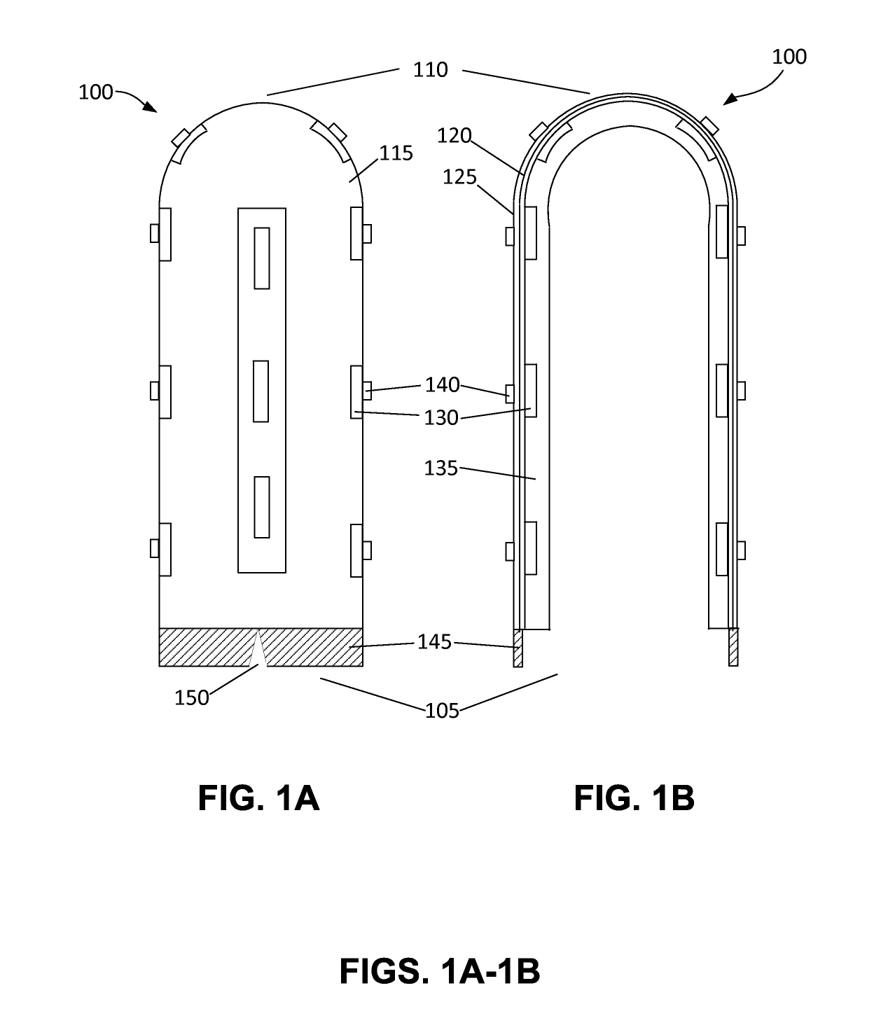

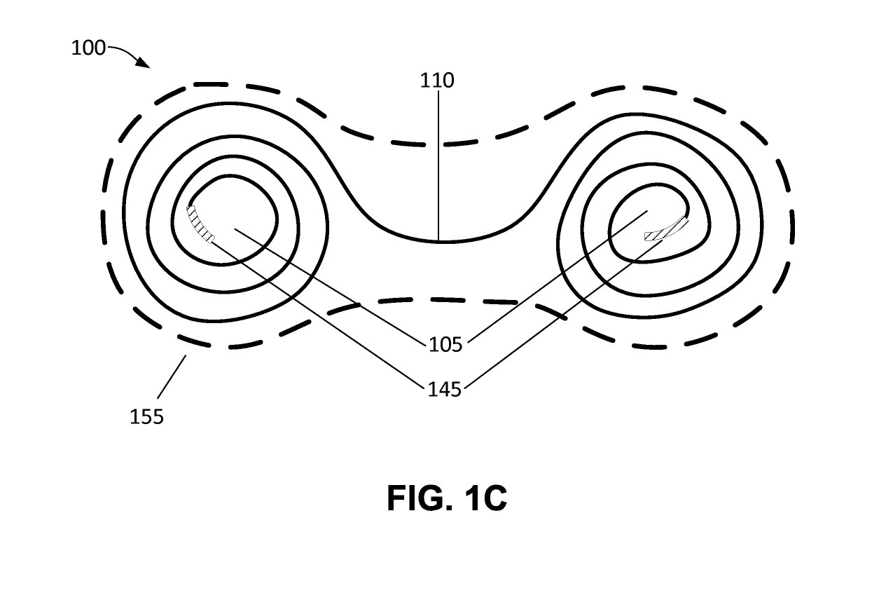

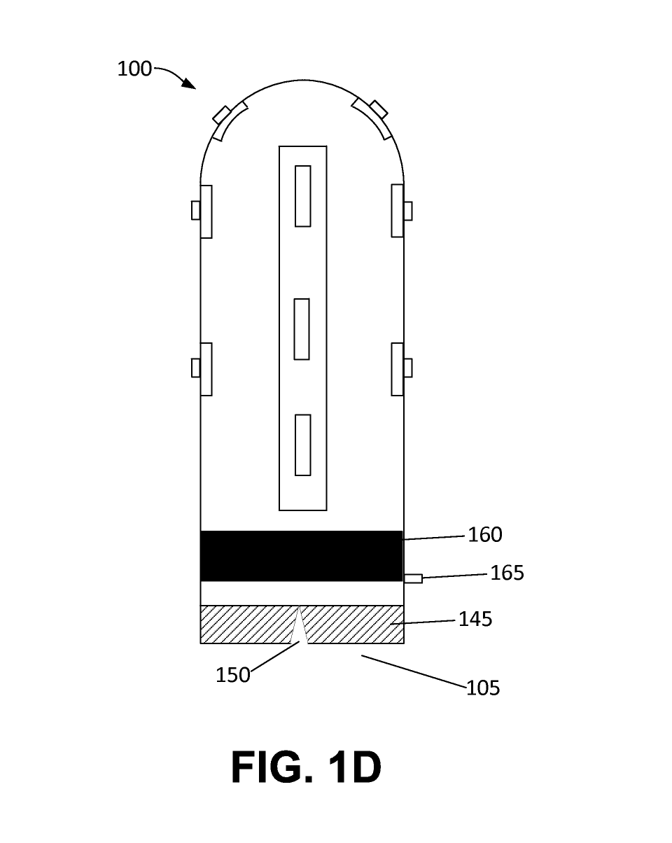

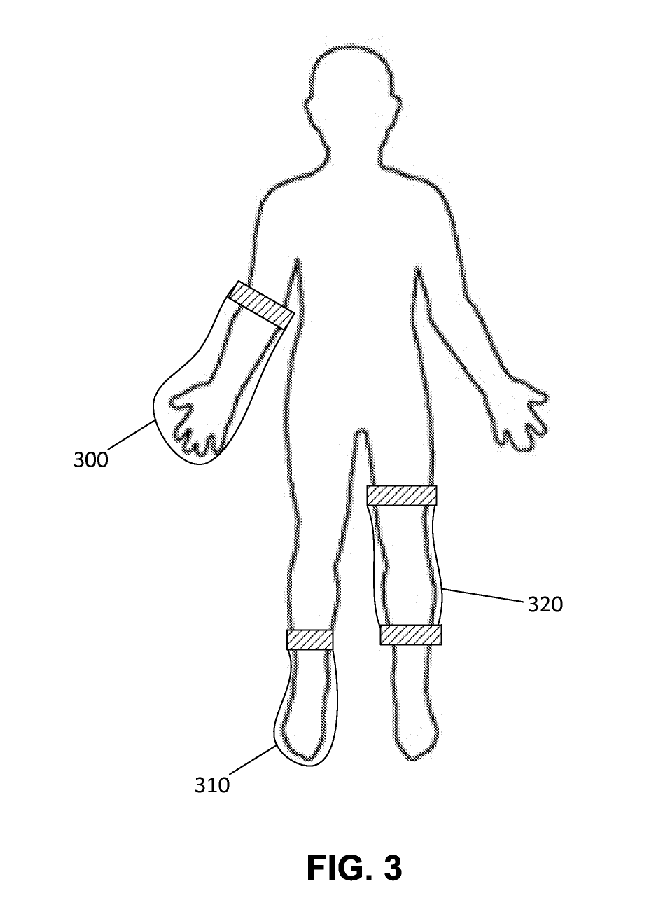

As illustrated in FIGS. 1A and 1B, the sterilization device 100 of the present disclosure is generally configured to have a tubular structure to facilitate placement over at least a portion of a subject's appendage. The sterilization device 100 may include an open end 105 in which the subject's appendage is initially inserted, and a closed end 110. The subject's appendage may be inserted into the open end 105 of the sterilization device 100 until the appendage is in close proximity to the closed end 110 of sterilization device 100. The sterilization device 100 may be applied by rolling the sterilization device 100 over the subject's appendage in a distal to proximal direction until the sterilization device 100 is in a fully deployed configuration, in which the open end 105 is proximal to the closed end 110 (see, e.g., arm embodiment in FIG. 3). Prior to deployment of the sterilization device 100 over the appendage of a subject, the sterilization device 100 is generally configured to be compressed or “rolled up,” as illustrated in FIG. 1C (e.g., doughnut shaped). In this configuration, the open end 105 of the sterilization device 100 (including the stabilization mechanisms 145) is at the center of each roll, while the closed end 110 occupies the middle area of the sterilization device 100. Sterile packaging 155 may enclose the rolled up sterilization device 100. The rolled up configuration of the sterilization device 100 facilitates convenient storage and transport of the sterilization device 100, and allows for efficient deployment of the sterilization device 100 over a subject's appendage.

The sterilization device 100 of the present disclosure may include more than one layer (e.g., it may be multilayered). One or more layers of the sterilization device 100 may be made from materials that are biocompatible, including, for example, materials derived from aliphatic urethanes, polyurethanes, polyesters, polyethers, amine modified polyethers and the like. The polymeric material used to construct one or more layers of the sterilization device 100 may also be linear and/or crosslinked biocompaitible, segmented polymers containing labile and/or biologically active moieties. The biocompatible polymers may also be based on biocompatible polyols or mixtures of polyols exhibiting various degrees of hydrophobicity. For example, polyurethane-based material may include cyclohexane dimethanol dimethacrylate, cyclohexane dimethanol diacrylate, alkoxylated hexanediol diacrylate, alkoxylated cyclohexane dimethanol diacrylate, caprolactone modified neopentylglycol hydroxypivalate diacrylate, ethylene glycol dimethacrylate, tetraethylene glycol dimethacrylate, polyethylene glycol dimethacrylate, 1,4-butanediol diacrylate, 1,4-butanediol dimethacrylate, diethylene glycol diacrylate, diethylene glycol dimethacrylate, 1,6-hexanediol diacrylate, 1,6-hexanediol dimethacrylate, neopentyl glycol diacrylate, neopentyl glycol dimethacrylate, polyethylene glycol diacrylate, tetraethylene glycol diacrylate, dipropylene glycol diacrylate, polyethylene glycol dimethacrylate, polyethylene glycol diacrylate, polyethylene glycol dimethacrylate water solution, propoxylated 2-neopentyl glycol diacrylate, and alkoxylated aliphatic diacrylate.

In some embodiments, one or more layers of the sterilization device 100 can be coated or treated with various substances to improve the effectiveness of the device. For example, the various layers of the sterilization device 100 can be coated with substances that help to prevent contamination from microorganisms, bacteria, fungi, viruses, and the like. The coatings can be active pharmaceutical agents that reduce the growth and/or survival of these harmful microorganisms (e.g., anti-bacterial substances), and/or the coatings can function passively to prevent contamination, for example, by preventing adherence of these microorganism to the various layers of the sterilization device 100 (e.g., wetting agents).

In another embodiment, sterilization device 100 includes a flexible sleeve 115 having inner layer 120 and an outer layer 125 (FIG. 1B). The inner layer 120 is made of a material that is substantially transparent to allow medical personnel to view the underlying portions of the sterilization device 100 (e.g., the foam insert 140), and impermeable to prevent leakage of the sterilization fluid and to maintain a sterile environment. In some cases, the inner layer 120 of sterilization device 100 can be made of polyurethane film that can be about 0.001 inches thick to about 0.01 inches thick. In some cases, inner layer 120 is made of a polyurethane film that is between about 0.001 inches thick and about 0.075 inches thick. In some cases, inner layer 120 can be made of a polyurethane film that is about 0.001 inches thick to about 0.005 inches thick. In some embodiments, inner layer 120 can be made of a polyurethane film that is about 0.002 inches thick to about 0.005 inches thick. In other embodiments, inner layer 120 can be made of a polyurethane film that is about 0.002 inches thick to about 0.04 inches thick. In yet other embodiments, inner layer 120 can be made of a polyurethane film that is about 0.004 inches thick to about 0.005 inches thick.

Outer layer 125 of flexible sleeve 115 of sterilization device 100 can be made of a material that is expandable and pliable to allow for application or positioning of the sterilization device 100 over the subject's appendage. Suitable materials for the outer layer 125 include, for example, fabric or fibrous material, similar to the material used for conventional surgical stockinettes. In certain embodiments, outer layer 125 can be made of a knit cotton yarn that is stretchable and elastic and forms a cross-linked mesh. Fibrous materials used to construct the outer layer can include a variety of natural and synthetic materials including, but not limited to, cotton, rayon, polyesters, polyolefins, polyamides, copolymers of the foregoing and generally any polymer or resin which can be drawn, extruded or otherwise formed into fibers.

In certain aspects, flexible sleeve 115 of sterilization device 100 can be made of a single layer. The single layer can be made of a polymeric material or a fibrous material, for example as described above. In other aspects, flexible sleeve 115 of sterilization device 100 can include two or more layers. In accordance with these aspects, the two or more layers may be made of polymeric material or a fibrous material, for example as described above. The number of layers making up the flexible sleeve 115 will generally depend on the particular medical procedure being performed, the setting or location in which the medical procedure is being performed, the characteristics of the subject's appendage, the characteristics of the subject's wound, and other relevant variables.

In one embodiment, sterilization device 100 can include a plurality of sterilization sacs 130 (FIGS. 1A and 1B). Sterilization sacs 130 can contain one or more sterilization agents to facilitate the sterilization of a subject's appendage prior to the commencement of a medical procedure. In some aspects, the sterilization agent can include hexidine, chlorhexidine betadine, iodine, alcohol, and/or derivatives and/or combinations thereof. Sterilization agents used herein may be in liquid form upon dispersal. The volumes and concentrations of these and other sterilization agents can readily be determined by one of skill in the art, and based on the present disclosure. In some cases, sterilization sacs 130 can include sterilization agents as well as other pharmaceutically active agents, including, for example, anesthetizing agents, anti-bacterial agents, anti-fungal agents and the like. The sacs containing pharmaceutically active agents, such as the sterilization sacs 130, may be made of a polymeric biocompatible material as described above, including, but not limited to, polymeric material derived from urethanes, polyurethanes, polyesters, polyethers, amine modified polyethers and the like.

Sterilization sacs 130, as well as sacs containing other pharmaceutically active agents, can be distributed throughout one more layers of the sterilization device 100. In some embodiments, the plurality of sterilization sacs 130 can be included within a layer of the sterilization device 100 that includes an absorbent foam insert 135 located underneath the transparent, flexible sleeve 115. The absorbent foam insert 135 can be the portion of the sterilization device 100 that is in direct contact with the skin and/or open wound of the subject's appendage. In some embodiment, the foam insert 135 is made of open cell foam. Typically, open cell foams are made up of interlocked or interconnected spaces that are filled with atmospheric air (similar to a sponge). The porous characteristics of open cell foams allow them to absorb liquid, including, for example, one or more sterilization agents contained within the sterilization sacs 130. In some cases, sacs containing other pharmaceutically active agents (e.g., anesthetizing agents, anti-bacterial agents, anti-fungal agents and the like) are also included within the foam insert 135. Open cell foams are made up of various materials (e.g. polyurethane). The sacs may be located in various positions throughout the foam insert 135, and may be used to deliver various medical treatments (e.g., anesthetization prior to surgery). The location of individual sacs, as well as the type of pharmaceutically active agent contained within it, may be communicated to medical personnel by various visual indicators. For example, different sacs containing different pharmaceutically active agents may be distinguished using words, symbols, colors, shapes, and/or textures. Clearly distinguished indicators may be especially important in emergency medical situations (e.g., on a battle field), where time is of the essence.

In another embodiment, sterilization device 100 can include a breaker structure and/or a mechanism 140 for rupturing one or more sacs containing the various pharmaceutically active agents and delivering the pharmaceutically active agent to the subject, as illustrated in FIGS. 1A and 1B. For example, the sterilization device 100 may include sterilization sacs 130 having one or more perforated breakers 140, or one or more areas where the material making up the sac is thinned compared to the other areas of the sac. The perforated breakers 140 may be located in the sterilization sac 130 such that the application of pressure ruptures the sterilization sac 130 and releases the sterilization agents.

In some cases, the sacs containing the pharmaceutically active agents, such as the sterilization sacs 130, are ruptured using a plurality of pointed breakers 140. For example, the outer layer 125 of the sterilization device 100 may include a plurality of pointed breakers 140 which are aligned with the underlying sterilization sacs 130 in the absorbent foam insert 135. A pointed breaker 140 may be any sharpened structure, such as a pin or needle, which can puncture a sterilization sac 130 upon the application of pressure, without compromising the integrity of the inner impermeable layer 120 of the sterilization device 100.

In other embodiments, the sacs containing the pharmaceutically active agents, such as the sterilization sacs 130, can include one or more valve-based breakers 140 arranged to separate the sterilization sacs 130 and release the pharmaceutically active agents upon the application of pressure. When valve breakers 140 are used, it is generally not necessary to rupture the sterilization sacs 130 to release the pharmaceutically active agents. For example, one or more valve breakers 140 may be coupled to the sterilization sacs 130. To release fluid from the sterilization sacs 130 and into the foam insert 135, the breaker valves 140 may be forced open by creating a pressure differential. A pressure differential can be created, for example, when a health professional applies mechanical pressure (e.g., squeezing or pressing) to the sterilization sac 130 or the area around the sterilization sac 130. A pressure differential can also be created, for example, using a source of compressed air or carbon dioxide. The compressed air or carbon dioxide may be injected into one or more sterilization sacs 130 to displace the fluid, causing the fluid to exit the sterilization sacs 130.

When a health professional applies pressure to or manipulate one more sacs containing a pharmaceutically active agent, such as a sterilization sac 130, and release the agents, the foam insert 135 absorbs the agent. In some cases, absorption of the agents into the foam insert 135 causes the foam insert 135 to change color or to become darker in color. This change in color or brightness in the foam insert 135 may be visualized by a health professional through the transparent sleeve 115, and may be an indication that the sterilization agents have successfully been applied to the subject's skin or wound area, according to some embodiments. For example, release of betadine or hexidine from a sterilization sac 130 may cause the foam insert 135 to change color from a light tan to a dark brown. A health professional may then apply mechanical force or pressure (e.g., mechanical scrub) to the foam insert 135 to distribute the sterilization agents over the skin of the subject's appendage to sterilize the appendage prior to the commencement of a medical procedure. The foam insert 135 may be sufficiently thick to absorb a suitable amount of the sterilization agents to ensure sterility of the surgical site. For example, the foam insert 135 may be about 0.25 inches thick to about 1 inch thick. In other embodiments, the foam insert 135 can be about 0.25 inches thick to about 0.75 inches thick. In yet other embodiments, the foam insert 135 can be about 0.25 inches thick to about 0.5 inches thick. In other aspects, the foam insert 135 can be about 0.5 inches thick.

In some embodiments, sterilization device 100 can include an array of circumferentially aligned anesthetic delivery devices for administering an anesthetizing agent to a subject prior to commencing a medical procedure. For example, the circumferentially aligned anesthetic delivery devices can include a plurality of small needles enclosed in protective housing that are exposed upon the application of pressure. When pressure is applied by a health profession, for example, the needles may be exposed and can deliver one or more anesthetizing agents to the subject. Health professionals have the ability to deliver as much anesthetic as believed to be necessary or appropriate for a given medical procedure by applying pressure to as many anesthetic delivery devices as desired. In some cases, the circumferentially aligned anesthetic delivery devices are located towards the open end 105 of the sterilization device 100 and contained within the foam insert 135. In other embodiments, the circumferentially aligned anesthetic delivery devices are located towards the open end 105 in an area of the sterilizing device 100 that does not include the foam insert 135.

Sterilization device 100 can also include one or more stabilization mechanisms 145 for both securing the sterilization device 100 to a portion of the subject's appendage and for creating a seal to maintain a sterile environment. The stabilization mechanism 145 is generally located at the open end 105 of the sterilization device 100, such that after the sterilization device 100 is “rolled over” the subject's appendage, the stabilization mechanism 145 can be used to secure the sterilization device 100 in a desired position. In some cases, the stabilization mechanism 145 includes one or more adhesive strips having a notch or slit 150 such that after the sterilization device 100 is in place, the adhesive portion of the strip is revealed and the two sides of the adhesive strip adjacent to the slit 150 contact the subject's skin in an overlapping manner. In some cases, the stabilization mechanism 145 includes adhesive strips, drawstrings, metal strips, combinations thereof, and/or other structures for sealing and securing a surgical stockinette.

In some embodiments, sterilization device 100 includes a plurality of stabilization mechanisms 145 located at the open end 105, the closed end 110, as well as in areas between the ends. One or more stabilization mechanisms 145, including, for example, pliable metal strips, may be located within one or more layers of the sleeve 115 such that the pliable metal strips can be crimped or formed over the subject's appendage prior to sterilization. In some cases, pliable metal strips may be inserted into the sterilization device 100 such that the pliable metal strips are able to bend or crimp around or in between the digits (e.g. the fingers and/or toes) of a subject's appendage. The use of stabilization mechanisms 145 in this way enhances stability and augments the application of the sterilization agents to the skin of the subject's appendage by helping to maintain contact between the foam insert 135 and the subject's skin.

In some embodiments, sterilization device 100 includes a tourniquet. For example, as illustrated in FIG. 1D, a tourniquet 160 may be coupled to the sterilization device 100 towards the open end 105 of the sterilization device 100. The tourniquet can include a bladder for receiving compressed air (e.g., carbon dioxide) and a valve 165 for inflating and deflating the bladder. The tourniquet can be coupled to the sterilization device 100 in a location that is distal to the stabilization mechanism 145 (FIG. 1D), or in a position that is proximal to the stabilization mechanism 145. The bladder of the tourniquet 160 may be inflated after the sterilization device 100 is positioned over the subject's appendage and secured using the stabilization mechanism 145. In some cases, the tourniquet may be used as the stabilization mechanism. The sterilization device 100 may be positioned over the subject's appendage, and inflation of the bladder of the tourniquet 160 can be used to secure and seal the sterilization device 100 to the subject's appendage. In some cases, tourniquet 160 can be coupled to one or more sacs containing pharmaceutically active agents, such that inflation of the bladder of the tourniquet supplies the pressure sufficient to rupture the sacs and deliver the agents to the foam insert 135 and/or to the subject's skin. The coupling of a tourniquet 160 to the sterilization device 100 enables a health professional to prep for and perform a medical procedure more efficiently, which can be especially crucial in an emergency medical situation (e.g., a battle field).

In some embodiments, sterilization device 100, the flexible sleeve 115, and the foam insert 135 can be coupled so that the plurality of breakers 140 in the outer layer 125 of the sleeve 115 align with the plurality of sterilization sacs 133 in the foam insert 135. In accordance with these embodiments, when the sterilization device 100 is positioned over the subject's appendage, there is generally no further adjustment performed to ensure that the breakers 140 align with the sterilization sacs 130. In other embodiments, the flexible sleeve 115 and the foam insert 135 may not be coupled before being positioned over the subject's appendage; the flexible sleeve 115 and the foam insert 135 may be separate portions of the sterilization device 100, and therefore, may be positioned separately on the subject's appendage. In accordance with these embodiments, the sleeve 115 may be positioned over the subject's appendage prior to inserting the foam insert 135 into the sleeve 115 and positioning the foam insert 135 such that the breakers 140 align with the sterilization sacs 130. In other embodiments, the foam insert 135 may be positioned over the subject's appendage prior to insertion into the sleeve 115 and aligning the breakers 140 with the sterilization sacs 130.

Embodiments of the present disclosure can also include kits that having one or more sterilization devices for sterilizing at least a portion of a subject's appendage. In accordance with these embodiments, a kit may include a sterilization device having a transparent, flexible sleeve with an outer layer having a plurality of breakers and an inner impermeable layer. Additionally, the sterilization kit may include an absorbent foam insert that includes a plurality of sterilization sacs, the sterilization sacs containing one or more sterilization agents. In some embodiments, the sleeve and the foam insert can be coupled such that the breakers align with the sterilization sacs prior to inclusion in the kit. In other cases, the sleeve and the foam insert are separate, uncoupled portions of the sterilization kit. The sterilization kit can also include one or more stabilization mechanisms for securing the sterilization device to at least a portion of a subject's appendage. The sterilization kit can also include a tourniquet coupled to a sterilization device, as well as a source of compressed air or carbon dioxide (e.g., a carbon dioxide cartridge) to inflate to a sufficient pressure (e.g., 250 mg Hg) the bladder of the tourniquet.

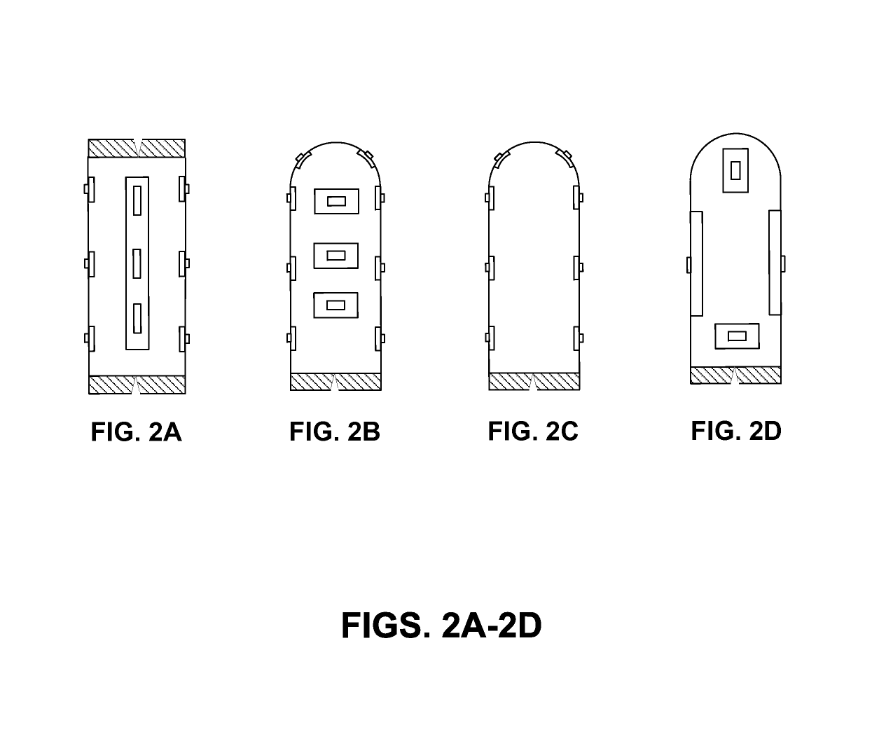

FIGS. 2A-2D depict other configurations of sterilization device 100. In some embodiments, sterilization device is open at both ends, as illustrated in FIG. 2A. In one embodiment, sterilization device 100 includes separate stabilization mechanisms 145 at either open end, such that the sterilization device 100 may be applied to a portion of a subject's appendage without enclosing the entire appendage within the sterilization device 100. Additionally, the stabilization mechanism 145 can include slits or notches 150 which overlap to secure and seal the subject's appendage within the sterilization device 100, as described above. In some embodiments, the sleeve 115 of the sterilization device 100 that is open at both ends is positioned over the subject's appendage prior to being secured and sealed. The foam insert 135 can then be inserted between the subject's appendage and the sleeve 115 such that the breakers 140 and the sterilization sacs 130 are aligned. After this alignment, the sterilization device can be secured and sealed to the subject's appendage using the stabilization mechanism 145.

Sterilization device 100 can include other arrangements of the breakers 140 and sterilization sacs 130, as illustrated in FIGS. 2B-2D. In some cases, one or more breakers 140 and sterilization sacs 130 are substantially horizontally aligned along the center of the sterilization device 100 (FIG. 2B). In other embodiments, sterilization device 100 can include one or more breakers 140 and sterilization sacs 130 on its periphery and not centrally located (FIG. 2C). In other embodiments, sterilization device 100 can include one or more enlarged breakers 140 and sterilization sacs 130 on its periphery and not centrally located (FIG. 2D). Various other configurations and sizes of breakers 140 and sterilization sacs 130 are contemplated herein, as can be readily determined by one of skill in the art based on the present disclosure and the particular medical procedure and the appendage to be treated.

FIG. 3 illustrates sterilization device 100 placed on various portions of a subject's appendages in order to facilitate the sterilization of that portion of the appendage prior to commencing a medical procedure. In some embodiments, the sterilization device 100 can be of a size suitable to cover the majority of a subject's appendage (e.g., passed the elbow or above the knee), as illustrated by device 300. In some embodiments, the sterilization device 100 can be a size suitable to cover a smaller portion of an appendage of a subject. For example, the sterilization device 100 can be sized to cover a subject's foot and ankle, as illustrated by device 310, or a subject's hand and wrist. In other aspects, where the sterilization device 100 is open at both ends, the sterilization device 100 can be configured to be of a size that covers a middle portion of a subject's appendage (e.g., the knee), without covering the rest of the appendage that is distal to the middle portion, as illustrated by device 320.

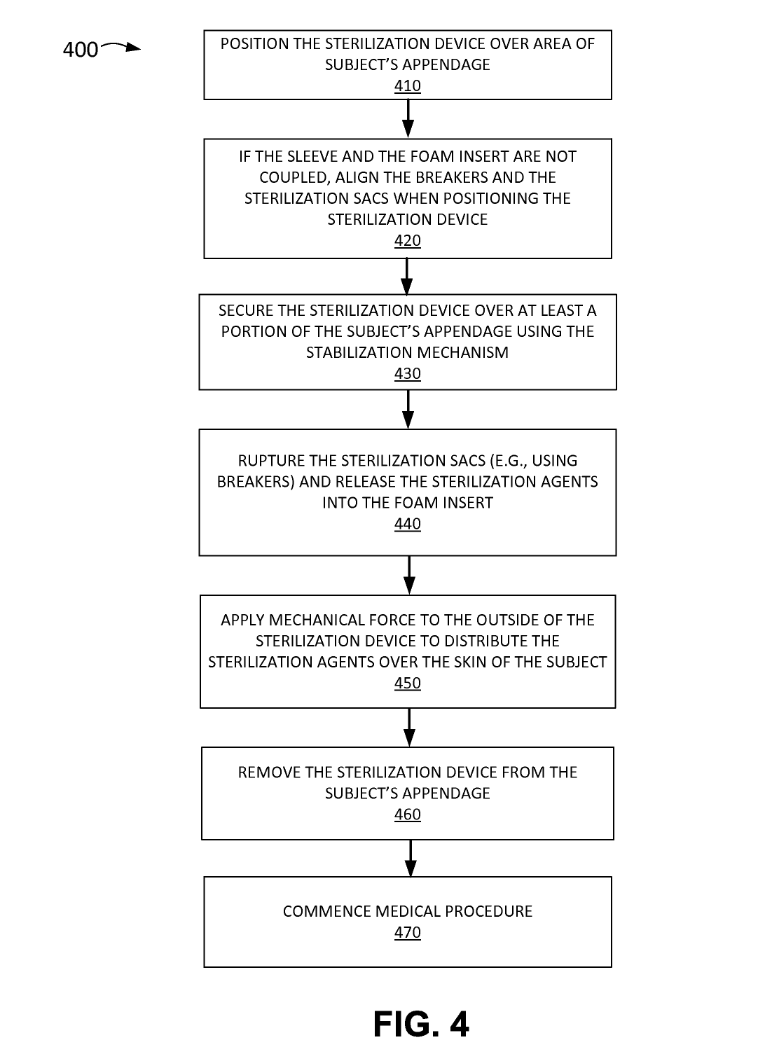

FIG. 4 illustrates a flowchart representing one method of sterilizing a portion of a subject's appendage, according to one embodiment. Generally, there are various steps that are taken before a medical procedure (e.g., a surgery) commences, including but not limited to anesthetizing a subject (often locally with the assistance of intravenous sedation), propping up the subject's appendage to hold the appendage away from any non-sterile environment, and preparing the subject's appendage using a variety of scrub brushes, sponges, and sterilization agents. This sterilization process can take at least as long as the time spent by a medical doctor (e.g., a surgeon) to scrub, and often times longer, because preparing the foot and leg, for example, is among many duties that medical personnel (e.g., prep nurse, physician's assistant and the like) often perform prior to the commencement of the medical procedure. In contrast, the sterilization device 100, 300 enables the medical doctor or clinician to sterilize the site to be operated on after donning surgical attire and allows other medical personnel to perform the other pre-surgery duties. Thus, the sterilization devices 100, 300 and additional embodiments described herein increase operating room efficiencies and reduce the cost of medical procedures, without increasing the risk of infection or otherwise jeopardizing patient health.

One method of sterilizing at least a portion of a subject's appendage can include positioning the sterilization device over the subject's appendage (block 410). If the sleeve and the foam insert are not already coupled, as described above, then the breakers and the sterilization sacs may be aligned, for example, prior to sterilization (block 420). After the sterilization device is in a desired position on the subject's appendage and the breakers are aligned with the sterilization sacs, the sterilization device is secured and sealed to the subject's appendage using one or more stabilization mechanisms (block 430). At block 440, the breakers are used to rupture the sterilization sacs and release the sterilization agents without causing leakage of the sterilization agents to the outside of the sterilization device. The sterilization agents are released into the foam insert, which absorbs the sterilization agents. At block 450, mechanical force (e.g., mechanical scrub) is applied to the portions of the foam insert that have absorbed the sterilization agents in order to distribute the sterilization agents over the subject's skin, thus sterilizing the appendage. At block 460, the sterilization device is removed (e.g., by cutting or unrolling) from the subject's appendage. The medical procedure is commenced (block 470).

Methods of sterilizing at least a portion of a subject's appendage using the sterilization devices described can be performed by anyone, for example, any health professional authorized to be in the operating room (e.g., surgeon, physician's assistant, nurse, aesthetician and the like). The method of sterilizing at least a portion of a subject's appendage can also be performed in an office setting as an out-patient or other minor medical procedure room without increasing the risk of infection or otherwise jeopardizing patient health, which further reduces the costs of performing the medical procedure. Additionally, the method of sterilizing at least a portion of a subject's appendage using the sterilization devices described can be performed in the context of an emergency medical situation, such as on a battle field or accident, where it is even more crucial to deliver treatment to a subject as quickly and efficiently as possible.

A number of variations and modifications of the disclosure can be used. It would be possible to provide for some features of the disclosure without providing others.

The present disclosure, in various aspects, embodiments, and configurations, includes components, methods, processes, systems and/or apparatus substantially as depicted and described herein, including various aspects, embodiments, configurations, subcombinations, and subsets thereof. Those of skill in the art will understand how to make and use the various aspects, aspects, embodiments, and configurations, after understanding the present disclosure. The present disclosure, in various aspects, embodiments, and configurations, includes providing devices and processes in the absence of items not depicted and/or described herein or in various aspects, embodiments, and configurations hereof, including in the absence of such items as may have been used in previous devices or processes, for example, for improving performance, achieving ease and/or reducing cost of implementation.

The foregoing discussion of the disclosure has been presented for purposes of illustration and description. The foregoing is not intended to limit the disclosure to the form or forms disclosed herein. Various features of the disclosure are grouped together herein in one or more, aspects, embodiments, and configurations for the purpose of streamlining the disclosure. The features of the aspects, embodiments, and configurations of the disclosure may be combined in alternate aspects, embodiments, and configurations other than those discussed above. This method of disclosure is not to be interpreted as reflecting an intention that the claimed disclosure requires more features than are expressly recited in each claim. Rather, as the following claims reflect, inventive aspects lie in less than all features of a single foregoing disclosed aspect, embodiment, or configuration. Thus, the following claims are hereby incorporated into this Detailed Description, with each claim standing on its own as a separate embodiment of the present disclosure.

Moreover, though the description of the present disclosure has included description of one or more aspects, embodiments, or configurations and certain variations and modifications, other variations, combinations, and modifications are within the scope of the disclosure, e.g., as may be within the skill and knowledge of those in the art, after understanding the present disclosure. It is intended to obtain rights which include alternative aspects, embodiments, and configurations to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A sterilization device for sterilizing an area of a subject's body, the sterilization device comprising:
a multilayered transparent sleeve comprising an outer flexible layer and an inner impermeable layer;
an absorbent foam insert comprising a plurality of sterilization sacs containing one or more sterilization agents, the absorbent foam insert positioned within the multilayered transparent sleeve and configured for placement against an area of a subject's body in need of medical treatment; and
one or more stabilization mechanisms for securing the sterilization device to the subject's body;
wherein at least one of the plurality of sterilization sacs is configured to release the one or more sterilization agents upon application of pressure to the sterilization sac onto the area of a subject's body in need of medical treatment.

2. The device of claim 1, wherein the inner impermeable layer is comprised of substantially transparent polymeric material.

3. The device of claim 1, wherein the inner impermeable layer is comprised of polyurethane.

4. The device of claim 1, further comprising a breaker configured to engage the sterilization sac upon application of pressure to the breaker to facilitate release of the one or more sterilization agents from the sterilization sac.

5. The device of claim 1, further comprising a breaker comprised of one or more structures selected from the group consisting of: a pointed structure, a perforation, and a valve.

6. The device of claim 1, wherein the absorbent foam insert is comprised of open cell foam.

7. The device of claim 1, wherein the one or more sterilization agents comprise one or more of hexidine, chlorhexidine, betadine, iodine, alcohol and derivatives and combinations thereof.

8. The device of claim 1, wherein the one or more stabilization mechanisms comprise one or more of adhesive strips, drawstrings, and metal strips.

9. The device of claim 1, wherein the multilayered transparent sleeve and the absorbent foam insert are coupled so that the breaker is positioned at least partially over the sterilization sac.

10. The device of claim 1, further comprising a tourniquet, the tourniquet comprising a bladder and a valve through which the tourniquet is inflated.

11. A method of sterilizing an area of a subject's appendage, the method comprising:
positioning a sterilization device over an area of the subject's appendage in need of medical treatment, the sterilization device comprising: a transparent sleeve having an outer layer and an inner impermeable layer; an absorbent foam insert comprising a plurality of sterilization sacs containing one or more sterilization agents; and one or more stabilization mechanisms;
securing the sterilization device to the area of the subject's appendage in need of medical treatment using the one or more stabilization mechanisms;
applying pressure to one or more of the plurality of sterilization sacs to release the one or more sterilization agents into the absorbent foam insert; and
applying mechanical force to the outer layer of the sterilization device to distribute the one or more sterilization agents through the foam insert to the area of the subject's appendage in need of medical treatment.

12. The method of claim 11, wherein the inner impermeable layer is comprised of polyurethane.

13. The method of claim 11, wherein pressure is applied to one or more breaker structures or mechanisms comprised of one or more of pointed structures, perforations, or valves to release the one or more sterilization agents from the plurality of sterilization sacs.

14. The method of claim 11, wherein the absorbent foam insert is comprised of open cell foam.

15. The method of claim 11, wherein the one or more sterilization agents comprise one or more of hexidine, chlorhexidine, betadine, iodine, and derivatives and combinations thereof.

16. The method of claim 11, wherein the sleeve and the foam insert are coupled so that a plurality of breakers in the outer layer of the sleeve align with the plurality of sterilization sacs in the foam insert.

17. The method of claim 11, wherein positioning the sterilization device further comprises aligning a plurality of breakers in the outer layer of the sleeve with the plurality of sterilization sacs in the foam insert.

18. The method of claim 11, further comprising inflating a bladder of a tourniquet that is coupled to the sterilization device.

19. A surgical sterilization kit comprising:
a multilayered transparent sleeve comprising an outer flexible layer and an inner impermeable layer;
an absorbent foam insert comprising a plurality of sterilization sacs, the sterilization sacs comprising one or more sterilization agents; and
one or more stabilization mechanisms for securing the sterilization device to an area of a subject's appendage in need of medical treatment,
wherein the application of pressure to the sterilization sacs releases the one or more sterilization agents from the plurality of sterilization sacs and sterilizes the area of the subject's appendage in need of medical treatment.

20. The kit of claim 19, further comprising a plurality of breaker structures or mechanisms comprised of one or more of pointed structures, perforations, or valves that facilitate the release of the one or more sterilization agents from the plurality of sterilization sacs.

21. The sterilization kit of claim 19, further comprising a tourniquet and a source of compressed air or carbon dioxide for inflating the tourniquet.
